(19) United States
(12) Patent Application Publication
(10) Pub. No.: US 2018/0223256 A1
(43) Pub. Date: Aug. 9, 2018

(54) MODIFIED T CELLS AND THEIR USES IN TREATING CANCER

(71) Applicant: UNIVERSITY OF ROCHESTER, Rochester, NY (US)

(72) Inventor: Minsoo Kim, Pittsford, NY (US)

(73) Assignee: UNIVERSITY OF ROCHESTER, Rochester, NY (US)

(21) Appl. No.: 15/893,047

(22) Filed: Feb. 9, 2018

Related U.S. Application Data
(60) Provisional application No. 62/456,827, filed on Feb. 9, 2017.

(51) Int. Cl. C12N 5/0783; C07K 14/705; A61P 35/00

(52) U.S. Cl. CPC C12N 5/0638; C07K 14/70517; C07K 14/70571; C07K 16/2809; C07K 2317/24; C07K 2317/92; A61P 35/00

(57) ABSTRACT

Provided herein are compositions and methods for treating cancer. The compositions include a modified T cell comprising a nucleic acid encoding a calcium translocating channelrhodopsin (CatCh) polypeptide. The methods comprise administering to the subject with cancer one or more modified T cells comprising a nucleic acid encoding a calcium translocating channelrhodopsin (CatCh) polypeptide and exposing the one or more modified T cells in the subject to a visible light source.

### CROSS-REFERENCE TO PRIORITY APPLICATIONS

This application claims priority to U.S. Provisional Application No. 62/456,827, filed Feb. 9, 2017, the entirety of which is incorporated by reference herein.

### STATEMENT REGARDING FEDERALLY FUNDED RESEARCH

This invention was made with Government Support under grant number CA194969 awarded by the National Institutes of Health. The Government has certain rights in the invention.

### BACKGROUND

Cancer is a group of diseases characterized by abnormal cell growth with the potential to invade or spread to other parts of the body. It is estimated that, by 2030, 21.7 million new cases of cancer and 13 million cancer-related deaths will occur worldwide. Therefore, methods and compositions for treating cancer are necessary.

### SUMMARY

Provided herein are compositions and methods for treating cancer. The compositions include a modified T cell comprising a nucleic acid encoding a calcium translocating channelrhodopsin (CatCh) polypeptide. The methods comprise administering to the subject with cancer one or more modified T cells comprising a nucleic acid encoding a calcium translocating channelrhodopsin (CatCh) polypeptide and exposing the one or more modified T cells in the subject to a visible light source.

### DESCRIPTION OF THE FIGURES

FIGS. 1a-1j show that regulatory T cells (Tregs) suppress CTL-mediated cytotoxicity by down-regulating intracellular calcium signals. FIG. 1(a) shows B16F10 melanoma cells intradermally injected into the ear skin. Tumor growth (bar) and Treg frequency (dot) were monitored. n>5 mice per group. FIG. 1(b) shows phenotypic characteristics of tumor infiltrating CD4⁺Foxp3⁺ Treg cells. FIG. 1(c) shows the results of a CTL-mediated tumor killing assay. OT-1 CTLs were incubated for 5 h with OVA peptide-loaded EL-4 cells at a 1:1 ratio in the presence of rTregs or activated aTregs. Apoptotic cells were stained for Annexin V (n=3). FIG. 1(d) and FIG. 1(e) show expression of perforin, granzyme B and CD107a on OT-1 CTLs incubated with OVA-pulsed EL-4 at a 1:1 ratio in the absence or presence of aTregs was measured by flow cytometry (n=3). FIG. 1(f) shows a Western blot analysis of TCR signaling proteins in OT-1 CTLs. OT-1 CTLs were sorted after incubation with OVA-loaded EL-4 cells in the absence or presence of aTregs for 5 h. FIG. 1(g) shows flow cytometry analysis of intracellular IP₃ receptor expression (left) and total IP production (right) measured in OT-1 CTLs (n=3). FIG. 1(h) shows maximum intracellular calcium release as measured by Fluo-4 intensity in OT-1 CTLs after stimulation with anti-CD3 and anti-CD28 Abs followed by additional ionomycin stimulation at 400 sec (OT-1 CTL alone, n=19; OT-1 CTL+aTreg, n=13). FIG. 1(i) shows a Western blot for NFAT1 phosphorylation (n=3) for the OT-1CTLs. FIG. 1(j) shows results of co-culturing OT-1 CTLs with aTreg in the absence or presence of SB431542 for 16 h. PKH-26 labeled OVA peptide-loaded EL-4 cells were added to the cells for 5 h and apoptotic EL-4 cells were stained for Annexin V (n=3). Throughout, data are the mean s.e.m. NS: non-significant, P<0.01; P<0.05, P<0.005; by two-tailed Student's t-test.

FIGS. 2a-2g show the effects of increased intracellular Ca²⁺ on CTL effector functions. FIGS. 2(a) and 2(b) show proliferation (CFSE) and expression of CD69 and CD25 on OT-1 CD8+ T cells after activation with SIINFEKL (SEQ ID NO: 19)(N4) peptide, SIIGFEKL (SEQ ID NO: 20) (G4) peptide, or PBS, in the absence or presence of ionomycin (n=3). FIGS. 2(c) and 2(d) show IFN-γ secretion (measured by ELISA) and CD107a expression (measured by flow cytometry) on OT-1 CD8+ T cells activated with N4 peptide, G4 peptide, or PBS, in the absence or presence of ionomycin (n=3). FIG. 2(e) show N4 peptide, G4 peptide, or PBS loaded target tumor cell killing in the presence of various concentrations of ionomycin (n=3). FIG. 2(f) shows inhibition of target tumor cell (N4-loaded) killing by aTreg in the absence or presence of ionomycin (n=3). FIG. 2(g) shows TGF-β1 secretion by aTreg with or without ionomycin treatment for 6 h, measured by ELISA of culture supernatants (n=3). Throughout, data are the mean±s e.m. For FIG. 2(b) & FIG. 2(e), data were analyzed with One-Way ANOVA with a Bonferonni post-test. For FIGS. 2(c), 2(d), 2(f), and 2(g) Data were analyzed with Student's t-test.

FIGS. 3a-3h show optical control of intracellular Ca²⁺ signal in CD8+ T cells. FIG. 3(a) shows representative fluorescence images of Fluo-4/AM-loaded HEK293 cells during light activation. Intensity traces of mock-transfected cells (Con; n=16) and cells transiently transfected with CatCh (n=20). FIG. 3(b) shows a Western blot analysis of NFAT1 phosphorylation in CatCh-expressing OT-1 CD8+ T cells after light stimulation for the indicated time. FIGS. 3(c)-3(d) show IFN-γ secretion and cytotoxicity of CatCh-expressing OT-1 CD8+ T cells in the presence of G4 peptide-loaded EL-4 cells with or without light stimulation (n=3). FIG. 3(e) shows killing of N4 peptide-loaded EL-4 cells after light stimulation of CatCh- or GFP-expressing OT-1 22 CD8+ T cells in the presence or absence of aTregs (n=3). FIG. 3(f) shows expression of perforin, granzyme B and CD107a on WT or Orai1-cKO CD8+ T cells after incubation with N4 peptide-loaded EL-4 cells at a 1:1 ratio for 6 h (n=3). FIG. 3(g) shows killing of G4 peptide loaded EL-4 cells by WT or Orai1-cKO CD8+ T cells (n=6). FIG. 3(h) shows killing of N4 peptide loaded EL-4 cells by CatCh-expressing WT or Orai1-cKO CD8+ T cells with light stimulation (463 nm) (n=3). Throughout, data are the mean±s.e.m. and were analyzed by two-tailed Student's t-test.

FIGS. 4a-4g show optical control of Ca²⁺ signal in adoptively transferred Pmel-1 T cells. FIG. 4(a) shows flow cytometry analysis of total T cell populations (day 13) in mice with B16 murine melanoma. Where indicated, mice were vaccinated by intradermal injection of hgp100/IFA. FIG. 4(b) shows experimental design of the studies of optical control of Ca²⁺ signal in vivo. FIG. 4(c) shows a freely moving mouse with battery-powered wireless LED attached on the ear skin. FIG. 4(d) shows size and growth curves of B16 tumors in mice treated with adoptive transfer of CatCh-expressing Pmel-1 CD8+ T cells with (optical LED+light) or without (optical LED+dark) light stimulation (light, n=6; dark, n=6). The light stimulation (470-nm) times are denoted with a green box. FIG. 4(e) shows size and growth curves of B16 tumors in mice treated with adoptive transfer of GFP-expressing Pmel-1 CD8+ T cells with (optical LED+light) or without (optical LED+dark) light stimulation (light, n=7; dark, n=6). The light stimulation (470-nm) times are denoted with a blue box. FIG. 4(f) shows flow cytometry analysis of total CD4+ T cells, CD8+ T cells, and CD4+ Foxp3+ Treg numbers (per 1×106 total cells) in B16 tumors on day 21 (7 days after light stimulation) (light, n=5; dark, n=8). FIG. 4(g) shows flow cytometry analysis of total Pmel-1 T cell number (per 1×10⁶ total cells) (light, 23 n=5; dark, n=8) and expression of IFN-γ B16 tumors, as analyzed by qPCR (n=3). Throughout, data are the mean±s.e.m. and were analyzed by two-tailed Student's t-test or One-Way ANOVA with a Bonferonni post-test.

FIG. 5 shows T cell infiltration in B16F10 melanoma. B16F10 melanoma cells were intradermally injected into the mouse ear skin. Tumor samples were collected on days 7, 14 and 21 (n>5). CD4⁺ Foxp3⁻ T cells, CD8+ T cells and CD4⁺Foxp3⁺ Treg cells were monitored by flow cytometry.

FIG. 6 shows treatment of OT-1 CTLs with ionomycin resulted in the dephosphorylation of NFAT1. OT-1 CTLs were stimulated by 1 μM ionomycin for indicated times, and were lysed in RIPA buffer. The cell lysates were analyzed by immunoblotting using anti-phospho-NFAT1 or anti-β-actin.

FIGS. 7a-7b show genotypic characterization and identification of CD4-cre Orai1 conditional knockout mice. FIG. 7(a) shows genomic DNA samples were prepared from the mice ears and purified CD8+ T cells, and PCR was performed using primers for the Cre gene sequence, intact Orai1-floxed allele and the recombined Orai1 allele. FIG. 7(b) shows mRNA expression level of Orai1 as analyzed by quantitative RT-PCR (n=3). Data were analyzed with Student's t-test.

FIG. 8 shows activation of WT and Orai1 cKO OT-1 CTLs after N4 peptide stimulation. Purified OT-1 CD8+ T cells were stimulated with N4 peptide and irradiated splenocytes. Expression of CD25 and CD69 were measured by flow cytometry.

FIGS. 9a-9d show battery powered LED operation and its electrical, optical, and thermal properties. FIG. 9(a) shows a scheme for a battery powered LED system. FIG. 9(b) shows light output current and voltage (LIV) measurement for a 505 nm LED. FIG. 9(c) shows temperature variation of the LED system for 90 second operation. FIG. 9(d) shows measured temperature distribution for the LED system at off and on (saturated) stage.

FIGS. 10a-f show optical control of of intracellular Ca²⁺ signal in CD8⁺ T cells overcomes immunosuppression. FIG. 10(a) shows that 2×10⁵ B16F10 cells in 10 μl PBS were intradermally injected into the ear and flank. Expression of CD107a on Pmel-1 CD8⁺ T cells in B16 tumors in the ear with (optical LED+light) or without (optical LED+dark) light stimulation of tumors in the ear (light, n=5; dark, n=5) is shown. FIG. 10(b) shows the size of B16 tumors both in the ear and flank treated with adoptive transfer of CatCh-expressing Pmel-1 CD8⁺ T cells with (optical LED+light) or without (optical LED+dark) light stimulation of the ear (light, n=5; dark, n=5). FIG. 10(c) shows expression of CD69 on OT-I CD8⁺ T cells after re-stimulation with N4 peptide (1 μg/ml) loaded irradiated splenocytes in the absence or presence of CGS21680 (10 μM, n=4). FIG. 10(d) shows expression of CD69 on OT-I CD8⁺ T cells after re-stimulation with N4 peptide (1 μg/ml) loaded irradiated splenocytes in the absence or presence of PGE₂ (5 n=4). FIG. 10(e) shows that OT-I CD8⁺ T cells were incubated with PBS or sFasL (100 ng/ml) for 16 h. Apoptotic OT-I CD8⁺ T cells were stained for Annexin V (n=4). FIG. 10(f) shows killing of N4 peptide-loaded EL-4 cells after light stimulation of CatCh- or GFP-expressing OT-I CD8⁺ T cells in the absence or presence of MDSC (1:1) (n=6).

FIG. 11(a) shows killing of hgp100₂₅₋₃₃ peptide-loaded B16F10 cells after light stimulation of CatCh- or GFP-expressing Pmel-1 CD8⁺ T cells for 16 h in the presence or absence of aTregs (n=4).

FIG. 11(b) shows growth curves of B16 tumors on the ear treated with adoptive transfer of CatCh-expressing Pmel-1 CD8⁺ T cells with vaccination with mgp100₂₅₋₃₃ (EGSRNQDWL) (light, n=5; dark, n=6). The light stimulation (470-nm) times are denoted with a box. Data were analyzed with Student's t-test.

### DETAILED DESCRIPTION

Adoptive cell transfer utilizing tumor-targeting cytotoxic T lymphocytes (CTLs) is used to treat hematological malignancies. However, significant clinical success has not yet been achieved in solid tumors due in part to the strong immunosuppressive tumor microenvironment. Overcoming this has to date been limited by non-specific stimulation of tumor growth, metastasis, and angiogenesis. Shown herein is that suppression of CTL killing by CD4⁺CD25⁺Foxp3⁺ regulatory T cell (Treg) is mainly mediated by TGFβ-induced inhibition of inositol trisphosphate (IP3) production, leading to a decrease in T cell receptor (TCR)-dependent intracellular Ca²⁺ response. Both in vitro and in vivo assays revealed that highly selective optical control of Ca²⁺ signaling in adoptively transferred modified CTLs was sufficient to overcome immunosuppression at the tumor site by enhancing T cell activation, IFN-γ production and antitumor cytotoxicity, leading to a significant reduction in tumor growth in mice. Together, these findings show that targeted optogenetic stimulation of intracellular Ca²⁺ signaling allows for the control of cytotoxic effector functions of adoptively transferred modified T cells with outstanding spatial resolution by boosting T cell immune responses only at the targeted tumor sites. Therefore, described herein are modified T cells for use in modulating tumor microenvironments and treating cancer in a subject.

Provided herein is a modified T cell comprising an exogenous nucleic acid encoding a calcium translocating channelrhodopsin (CatCh) polypeptide. The modified T cell or T lymphocyte provided herein can be a non-regulatory CD4⁺ T cell or a cytotoxic T lymphocyte, also known as a CTL; a CD8+ T cell; a killer T cell; a T-killer cell; a cytotoxic T cell; or a T_{c}. Modified natural killer (NK) cells can also be used. In some examples, the modified T cell is an activated modified T cell. The modified T cell can be a modified T cell differentiated from a modified precursor cell comprising an exogenous nucleic acid encoding a CatCh polypeptide. Therefore, also provided are modified precursor cells that can be differentiated into T cells, wherein the modified precursor cell comprises an exogenous nucleic acid encoding a CatCh polypeptide. The precursor cells can be, for example, pluripotent stem cells, including induced pluripotent stem cells. Methods of isolating precursors and making induced pluripotent stem cells are known in the art. See, for example, Hanna et al. Science 318(5858): 1920-23 (2007), incorporated herein in its entirety by this reference. For example, the cell can be a CD34+ cell comprising a nucleic acid encoding a CatCh polypeptide that can, under proper conditions, differentiate into a T cell. The CD34+ cell can be selected from the group consisting of a primary CD34+ hematopoietic progenitor cell, a CD34+ peripheral blood cell, a CD34+ cord blood cell and a CD34+ bone marrow cell.

The modified T cells are not cancer cells, tumor cells or transformed cells. To confirm, cells can be screened before and/or after modification with a nucleic acid encoding a CatCH polypeptide for evidence of undesirable genetic characteristics. The modified T cells can also be screened before or after administration to a subject.

The cell can be in vitro, ex vivo or in vivo. Thus, the modified cells is optionally present in a heterologous medium.

The modified T cells described herein can further comprise a nucleic acid encoding a chimeric antigen receptor (CAR). For example, chimeric antigen receptor (CAR) sequences can be introduced into a T cell or a T cell precursor in order to generate cancer specific T cells which target cancer cells.

Any of the nucleic acids described herein can be introduced into T cells or precursors thereof by well-known methods. These methods include, but are not limited to, viral transduction (for example, Frecha et al. Mol. Ther. 18(10): 1748-57 (2010), lipofection, electroporation, liposomal delivery, sonoporation, CRISPR/Cas9-mediated insertion, and nucleoporation. Methods for nucleoporation are known in the art. See, for example, Maasho et al. “Efficient gene transfer into the human natural killer cell line, NKL, using the amaxa nucleofection system,” Journal of Immunological Methods 284(1-2): 133-140 (2004); and Aluigi et al. “Nucleofection is an efficient non-viral transduction technique for human bone marrow derived mesenchymal stem cells,” Stem Cells 24(2): 454-461 (2006)), both of which are incorporated herein in their entireties by this reference. RNA electroporation using in vitro-transcribed RNA can also be used to transfect T lymphocytes or precursors thereof. See, for example, Zhao et al. “High-efficiency Transfection of Primary Human and Mouse T Lymphocytes Using RNA Electroporation,” Mol. Ther. 13(1): 151-159 (2006), incorporated herein in its entirety by this reference. The present disclosure includes all forms of nucleic acid delivery, including naked DNA, plasmid and viral delivery, integrated into the genome or not.

A vector comprising a nucleic acid sequence encoding a CatCh polypeptide is also provided. The vector can direct the in vivo or in vitro synthesis of any of the CatCh polypeptides or fragments thereof described herein. The vector is contemplated to have the necessary functional elements that are functionally coupled to direct and regulate transcription of the inserted nucleic acid. These functional elements include, but are not limited to, a promoter; regions upstream or downstream of the promoter, such as enhancers that can regulate the transcriptional activity of the promoter; an origin of replication; appropriate restriction sites to facilitate cloning of inserts adjacent to the promoter; antibiotic resistance genes or other markers that can serve to select for cells containing the vector or the vector containing the insert, RNA splice junctions, a transcription termination region, or any other region that can serve to facilitate the expression of the inserted nucleic acid. See generally, Sambrook et al., Molecular Cloning: A Laboratory Manual. 2nd Ed., Cold Spring Harbor Laboratory, Cold Spring Harbor, N.Y. (1989). The vector, for example, can be a plasmid. The vectors can contain genes conferring hygromycin resistance, ampicillin resistance, gentamicin resistance, neomycin resistance or other genes or phenotypes suitable for use as selectable markers, or methotrexate resistance for gene amplification.

Viral vectors comprising the nucleic acids are also provided. For example, the nucleic acids can be in an adenoviral vector, an adeno-associated virus vector, an alphavirus vector, a herpesvirus vector, a lentiviral vector, a retroviral vector or a vaccinia virus vector, to name a few.

The expression vectors described herein can optionally include nucleic acids encoding a CatCh polypeptide under the control of an inducible promoter such as the tetracycline inducible promoter or a glucocorticoid inducible promoter. The nucleic acids disclosed herein can optionally be under the control of a tissue-specific promoter to promote expression of the nucleic acid in specific cells, tissues or organs. For example, the nucleic acid can be under the control of a promoter that promotes expression in a T cell or a precursor thereof. Any regulatable promoter, such as a metallothionein promoter, a heat-shock promoter, and other regulatable promoters, of which many examples are known in the art are also contemplated. Furthermore, a Cre-loxP inducible system can also be used, as well as the Flp recombinase inducible promoter system.

As used herein, a CatCh polypeptide is a photoactivatable membrane polypeptide or a fragment thereof that functions as a light-gated calcium channel. Wild-type CatCh is a transmembrane protein that contains a covalently linked retinal chromophore. Upon activation, or light absorption, the retinal chromophore undergoes a conformational change that opens the ion channel and allows the influx of extracellular calcium ions through the channel. In some examples, the CatCh polypeptide comprises SEQ ID NO: 1 or a functional fragment thereof. SEQ ID NO: 1 is full-length CatCh polypeptide and a non-naturally occurring variant of the wild-type channelrhodopsin 2 (ChR2) sequence expressed in Chlamydomonas reinhardtii, a single-cell green algae. A full-length CatCh polypeptide comprises seven transmembrane helices. However functional fragments of a CatCh polypeptide comprising at least five transmembrane helices, six transmembrane helices or seven transmembrane helices are also provided herein. As used herein, a functional fragment is a fragment of a CatCh polypeptide that forms a channel, and upon photoactivation, facilitates Ca²⁺ influx through the channel. A functional fragment of a CatCh polypeptide does not have to retain 100% activity as compared to the full-length wild-type CatCh polypeptide, as functional fragments that retain at least 60%, 70%, 80%, 90%, 95% activity or greater can also be expressed in the modified T cells provided herein.

Variants of the nucleic acids and polypeptides set forth herein are also contemplated. Variants typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identity to the wild type sequence and retain at least one function of the wild type sequence, for example, photoactivatable calcium channel activity. Those of skill in the art readily understand how to determine the identity of two polypeptides or nucleic acids. For example, the identity can be calculated after aligning the two sequences so that the identity is at its highest level.

Another way of calculating identity can be performed by published algorithms. Optimal alignment of sequences for comparison can be conducted using the algorithm of Smith and Waterman Adv. Appl. Math. 2: 482 (1981), by the alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.; the BLAST algorithm of Tatusova and Madden FEMS Microbiol. Lett. 174: 247-250 (1999) available from the National Center for Biotechnology Information (http://www.ncbi.nlm.nih.gov/blast/b12seq/b12.html), or by inspection.

The same types of identity can be obtained for nucleic acids by, for example, the algorithms disclosed in Zuker, M. Science 244:48-52, 1989; Jaeger et al. Proc. Natl. Acad. Sci. USA 86:7706-7710, 1989; and Jaeger et al. Methods Enzymol. 183:281-306, 1989 that are herein incorporated by this reference for at least material related to nucleic acid alignment and identity calculation. It is understood that any of the methods typically can be used and that, in certain instances, the results of these various methods may differ, but the skilled artisan understands, if identity is found with at least one of these methods, the sequences would be said to have the stated identity.

For example, as used herein, a sequence recited as having a particular percent identity to another sequence refers to sequences that have the recited identity as calculated by any one or more of the calculation methods described above. For example, a first sequence has 80 percent identity, as defined herein, to a second sequence if the first sequence is calculated to have 80 percent identity to the second sequence using the Zuker calculation method even if the first sequence does not have 80 percent identity to the second sequence as calculated by any of the other calculation methods. As yet another example, a first sequence has 80 percent identity, as defined herein, to a second sequence if the first sequence is calculated to have 80 percent identity to the second sequence using each of calculation methods (although, in practice, the different calculation methods will often result in slightly different calculated identity percentages).

As used herein, photoactivatable means that the CatCh polypeptide is activated by light. For example, the photoactivatable polypeptides described herein can be activated at wavelengths from about 450 nm to about 515 nm. In some examples, an implantable optogenetic stimulation device can be implanted in the subject, for example, in an organ of a subject, to deliver light to a tumor established in a subject's tissues and activate any of the modified T cells described herein at a tumor site. See, for example, Montgomery et al. “Wirelessly powered, fully internal optogenetics for brain, spinal and peripheral circuits in mice,” Nature Methods 12: 969-974 (2015); and Kim et al. “Injectable, cellular-scale optoelectronics with applications for wireless optogenetics,” Science 340: 211-218 (2013).

Also provided is a subject comprising a modified T cell comprising an exogenous nucleic acid that encodes a CatCh polypeptide or a fragment thereof, wherein the modified T cell expresses the CatCh polypeptide or a fragment thereof. The subject can be a mammal such as a primate, e.g., a human or a non-human primate. Non-human primates include marmosets, monkeys, chimpanzees, gorillas, orangutans, and gibbons, to name a few. Domesticated animals, such as cats, dogs, etc., livestock (for example, cattle (cows), horses, pigs, sheep, goats, etc.), laboratory animals (for example, ferret, chinchilla, mouse, rabbit, rat, gerbil, guinea pig, etc.) are also included. Thus, veterinary uses are also provided herein. The term subject does not denote a particular age or sex. Thus, adult and newborn subjects, whether male or female, are intended to be covered. As used herein, patient or subject may be used interchangeably and can refer to a subject afflicted with a disease or disorder.

Provided herein are methods of making one or more T cells or precursors thereof transduced or transfected with an exogenous nucleic acid encoding a CatCh polypeptide, optionally, prior to administration to an animal. The modified T cells can be autologous cells or heterologous T cells or precursor thereof. Autologous cells, i.e., a cell or cells taken from the same subject in need of treatment can be used to avoid immunological reactions that can result in rejection of the cells. In other words, when using autologous T cells or precursors thereof, the donor and recipient are the same subject. Alternatively, the T cells or precursors thereof can be heterologous, e.g., taken from a donor, preferably a related donor. The second subject (i.e., recipient) can be of the same or different species as the donor. Typically, when the cells come from a donor, they will be from a donor who is sufficiently immunologically compatible with the recipient to reduce the chances of transplant rejection, and/or to reduce the need for immunosuppressive therapy. The cells can also be obtained from a xenogeneic source, i.e., a non-human mammal that has been genetically engineered to be sufficiently immunologically compatible with the recipient, or the recipient's species.

Further provided is a transgenic non-human animal and methods of making a transgenic non-human animal, wherein the genome of the animal comprises an exogenous nucleic acid encoding a CatCh polypeptide described herein. As discussed above, the nucleic acid can be operably linked to a cell-specific or tissue specific promoter. The transgenic animal can be made by methods known in the art. For the purposes of generating a transgenic animal, screening the transgenic animal for the presence of a transgene and other methodology regarding transgenic animals, please see U.S. Pat. Nos. 6,111,166; 5,859,308; 6,281,408 and 6,376,743, which are incorporated by this reference in their entireties. For example, the transgenic animals can be made by (a) injecting a transgene comprising a nucleic acid encoding a CatCh polypeptide linked to an expression sequence into an embryo and (b) allowing the embryo to develop into an animal. The method can further comprise crossing the animal with a second animal to produce a third animal (progeny). Cells comprising a transgene, wherein the transgene comprises a nucleic acid encoding a CatCh polypeptide can be isolated from the transgenic animal. The transgenic animal includes, but is not limited to, mouse, rat, rabbit or guinea pig.

In the transgenic animals described herein, the transgene can be expressed in a specific cell type, for example, a T cell or a precursor thereof. Therefore, a T cell specific expression sequence can be selected such that expression of the transgene is primarily directed to T cells, but not exclusively to T cells. It is understood that when the transgene is primarily directed to T cells, some expression of the transgene, for example, 10% or less, can occur in other cells.

In the transgenic animal disclosed herein, expression of the transgene can be controlled by an inducible promoter. The transgenic animal of this invention can utilize an inducible expression system such as the cre-lox, metallothionine, or tetracycline-regulated transactivator system. An example of the cre-lox system for inducible gene expression in transgenic mice was published by R. Kuhn et al., “Inducible gene targeting in mice,” Science, 269(5229): 1427-1429, (1995) which is incorporated in its entirety by this reference. Use of the tetracycline inducible system is exemplified in D. Y. Ho et al., “Inducible gene expression from defective herpes simplex virus vectors using the tetracycline-responsive promoter system,” Brain Res. Mol. Brain. Res. 41(1-2): 200-209, Sep. 5, 1996; Y. Yoshida et al., “VSV-G-pseudotyped retroviral packaging through adenovirus-mediated inducible gene expression,” Biochem. Biophys. Res. Commun. 232(2): 379-382, Mar. 17, 1997; A. Hoffman et al., “Rapid retroviral delivery of tetracycline-inducible genes in a single autoregulatory cassette,” PNAS, 93(11): 5185-5190, May 28, 1996; and B. Massie et al., “Inducible overexpression of a toxic protein by an adenovirus vector with a tetracycline-regulatable expression cassette,” J. Virol. 72(3): 2289-2296, March 1998, all of which are incorporated herein in their entireties by this reference.

Also provided is a method of treating cancer in a subject comprising administering to the subject one or more modified T cells or precursors thereof comprising an exogenous nucleic acid encoding a CatCh polypeptide and exposing the one or more modified T cells in the subject to a visible light source.

One or more T cells can be transduced or transfected with an exogenous nucleic acid encoding a CatCh polypeptide or a fragment thereof prior to administration to the subject. Optionally, any of the T cells or precursors thereof described herein can comprise multiple copies of a nucleic acid encoding a CatCh polypeptide. Alternatively, a T cell precursor can be transduced or transfected with an exogenous nucleic acid encoding a CatCh polypeptide or a fragment thereof, followed by differentiation of the precursor cell into one or more T cells, prior to administration of the one or more T cells to the subject. The modified T cells can be autologous T cells or heterologous T cells. Any of the methods of treating cancer described herein can further comprise administering one or more immunosuppressants to the subject.

In any of the methods provided herein, the one or more T cells can be activated or expanded prior to administration to the subject. For example, the one or more modified T cells can be activated by contacting the one or more modified T cells in vitro with an anti-CD3 and/or an anti-CD28 antibody.

In any of the methods described herein, about 10³ to 10⁸ modified T cells can be administered to the subject, including 10³ to 10⁵, 10⁵ to 10⁸, 10⁴ to 10⁷ cells or any amount in between in total for an adult subject. This method can optionally comprise the step of diagnosing a subject with cancer.

In any of the methods provided herein, the visible light source can be any source that emits light in the visible light spectrum, for example, a laser, an optical fiber or a light emitting diode. In the methods set forth herein, Ca²⁺ influx through the CatCh polypeptide on the surface of modified T cells can be induced by exposing the cells to a visible light source that emits light, for example, at a wavelength of about 450 to 515 nm. Methods for assessing light-mediated influx of Ca²⁺ ions through a CatCh polypeptide in modified T cells in vitro and in vivo are described in the Examples. The cells can be exposed to a timed pulse(s) of light, for example, a pulse(s) of about 15 seconds, 20 seconds, 25 seconds, 30 seconds, 35 seconds 40 seconds or any amount of time in between. The cells can also be continuously exposed to the light source, for example, for about less than an hour, 1 hour, 2 hours, 3 hours, 4 hours, 5 hours, 10 hours, 15 hours, 20 hours, 25 hours, 30 hours, 35 hours, 40 hours or any amount of time in between. Each light exposure cycle can be repeated for multiple days and weeks, if necessary. If timed pulses are employed, one of skill in the art can determine how long each pulse should be and how long the interval between pulses should be. One of skill in the art can also determine whether single or multiple exposures to light are necessary. Exposure times and wavelengths can be determined empirically by exposing the modified T cells to the visible light source, assessing T cell activity, for example, calcium influx, and adjusting the exposure time, number of pulses, and/or wavelength accordingly.

After one or more cells are administered to the subject, the one or more cells are exposed to a visible light source, for example, at a tumor site. As set forth above, the visible light source can be a laser, an optical fiber or a light emitting diode. If the subject has skin cancer, the cells can be delivered to the subject, for example, by local injection or transdermally, prior to exposing the target of the subject's skin to the visible light source. The cells can also be delivered to a subject intrarectally, for example to treat colon or rectal cancer; intractracheally/intrabronchially, for example to treat lung cancer; laproscopically, for example, to treat liver, pancreatic, or kidney cancer; or intravaginally, for example, to treat cervical or uterine cancer, followed by exposure of the cells to a visible light source via endoscopic methods. In the methods set forth herein, the cells can also be administered to a tumor in the subject or at a surgical site followed by exposure to visible light, for example, via laser or endoscopic methods. Cannulation can also be utilized to insert an optical fiber at a desired site.

In the methods provided herein, exposing one or more modified T cells comprising a nucleic acid encoding a CatCh polypeptide to visible light can increase the immune response of the subject, promote T cell-mediated tumor killing and/or selectively stimulates Ca²⁺ production in the one or more modified T cells. By selectively activating Ca²⁺ production in the one or more modified T cells, immunosuppressive or regulatory T cells are not activated in the subject, thus overcoming regulatory T cell (T_{reg})-mediated immunosuppression. Therefore, by selectively delivering Ca²⁺ activation signals only to adoptively transferred modified CTLs in vivo, without interfering with endogenous Ca²⁺ signals in other cell types in the tumor microenvironment, cancer can be treated.

As used throughout, by subject is meant an individual. For example, the subject is a mammal, such as a primate, and, more specifically, a human. Non-human primates are subjects as well.

As used herein, cancer can be, but is not limited to, neoplasms, which include solid and non-solid tumors. A neoplasm can include, but is not limited to, pancreatic cancer, breast cancer, head and neck cancer, ovarian cancer, melanoma, bladder cancer, bone cancer, brain cancer (e.g., glioblastoma or neuroblastoma), lung cancer, prostate cancer, colon cancer, cervical cancer, esophageal cancer, endometrial cancer, central nervous system cancer, gastric cancer, colorectal cancer, thyroid cancer, renal cancer, oral cancer, Hodgkin lymphoma, skin cancer, adrenal cancer, liver cancer, and leukemia.

As used herein, the terms treatment, treat, treating or ameliorating refers to a method of reducing one or more effects of a disease or condition or one or more symptoms of the disease or condition, including a recurrence of the disease or condition. Thus in the disclosed method, treatment can refer to a 10%, 20%, 30%, 40%, 50%, 60%, 70%, 80%, 90%, or 100% reduction or amelioration in the severity of an established disease or condition or symptom of the disease or condition, and can refer to a 10%, 20%, 30%, 40%, 50% 60%, 70%, 80%, 90%, or 100% increase in survival time. For example, the method for treating cancer is considered to be a treatment if there is a 10% reduction in one or more symptoms of the disease in a subject as compared to control. For example, and not to be limiting, the method for treating cancer is considered to be a treatment if there is a 10% reduction in tumor size in a subject as compared to control or a 105 increase in survival time. Thus, the change can be a 10, 20, 30, 40, 50, 60, 70, 80, 90, 100%, or any percent reduction in between 10 and 100 as compared to, for example, a subject that does not receive one or more modified T cells comprising an exogenous nucleic acid encoding a CatCh polypeptide. It is understood that treatment does not necessarily refer to a cure or complete ablation of the disease, condition, or symptoms of the disease or condition.

The methods of treating cancer can optionally further comprise adminsitering another anti-cancer therapy, for example, surgery, radiation therapy or chemotherapy. Optional anti-cancer treatments can be administered prior to, concurrently with or subsequent to administration of the cells.

As used throughout, chemotherapeutic agents are compounds that inhibit the growth of cancer cells or tumors. Optionally, one or more chemotherapeutic agents can be used in any of the methods set forth herein. For example, two or more chemotherapeutic agents, three or more chemotherapeutic agents, four or more chemotherapeutic agents, etc. can be used in the methods provided herein. The chemotherapeutic agents that can be used include, but are not limited to, antineoplastic agents such as Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; 5-Fluorouracil; Flurocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin C; Mitosper; Mitotane; Mitoxantrone; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; and Zorubicin Hydrochloride.

Compositions comprising the modified T cells described herein can be prepared by making a cell suspension of the modified cells in a culture medium or a pharmaceutically acceptable carrier. Thus, provided herein is a pharmaceutical composition comprising an effective amount of the modified T cells in a pharmaceutically acceptable carrier. The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, dextrose, and water.

An agent or agents delivered in combination with the cells can be administered in vitro or in vivo in a pharmaceutically acceptable carrier. A pharmaceutically acceptable carrier for the agent can be a solid, semi-solid, or liquid material that can act as a vehicle, carrier or medium. Thus, compositions can be in the form of tablets, pills, powders, lozenges, sachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

Some examples of suitable carriers include phosphate-buffered saline or another physiologically acceptable buffer, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. A pharmaceutical composition additionally can include, without limitation, lubricating agents such as talc, magnesium stearate, and mineral oil; wetting agents; emulsifying and suspending agents; preserving agents such as methyl- and propylhydroxy-benzoates; sweetening agents; and flavoring agents. Pharmaceutical compositions can be formulated to provide quick, sustained or delayed release after administration by employing procedures known in the art. In addition to the representative formulations described below, other suitable formulations for use in a pharmaceutical composition can be found in Remington: The Science and Practice of Pharmacy 22d edition Loyd V. Allen et al., editors, Pharmaceutical Press (2012).

Liquid formulations for oral administration or for injection of one or more agents described herein generally include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles. Compositions for inhalation include solutions and suspensions in pharmaceutically acceptable, aqueous or organic solvents, or mixtures thereof, and powders. These liquid or solid compositions may contain suitable pharmaceutically acceptable excipients as described herein. Such compositions can be administered by the oral or nasal respiratory route for local or systemic effect. Compositions in pharmaceutically acceptable solvents may be nebulized by use of inert gases. Nebulized solutions may be inhaled directly from the nebulizing device or the nebulizing device may be attached to a face mask tent or intermittent positive pressure breathing machine. Solution, suspension, or powder compositions may be administered, orally or nasally, from devices which deliver the formulation in an appropriate manner. Another formulation that is optionally employed in the methods of the present disclosure includes transdermal delivery devices (e.g., patches). Such transdermal patches may be used to provide continuous or discontinuous infusion of an agent described herein.

According to the methods taught herein, the subject is administered an effective amount of modified T cells. The terms effective amount and effective dosage are used interchangeably. The term effective amount is defined as any amount necessary to produce a desired physiologic response. Effective amounts and schedules for administering the cells can be determined empirically, and making such determinations is within the skill in the art. The dosage ranges for administration are those large enough to produce the desired effect (e.g., a decrease in tumor size or increased survival time of a subject) in which one or more symptoms of the disease or disorder are affected (e.g., reduced or delayed). The dosage should not be so large as to cause substantial adverse side effects, such as unwanted cross-reactions, anaphylactic reactions, and the like. The effects will vary with e.g., the species, age, body weight, general health, sex and diet of the subject, the mode and time of administration, rate of excretion, treatment combination, and severity of the particular condition and can be determined by one of skill in the art. As discussed above, the amount of modified T cells can be from 10³ to 10⁸, for example. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

Any appropriate route of administration may be employed, for example, parenteral, intravenous, subcutaneous, intramuscular, intraventricular, intracorporeal, intraperitoneal, or rectal administration. Administration can be systemic (e.g., intravenous) or local (e.g., directly into a tumor). Pharmaceutical compositions can be delivered locally to the area in need of treatment, for example by topical application or local injection. Multiple administrations and/or dosages can also be used. Effective doses can be extrapolated from dose-response curves derived from in vitro or animal model test systems.

The disclosure also provides a pharmaceutical pack or kit comprising one or more containers filled with modified T cells or precursors thereof and optionally with one or more additional reagents or therapeutic agents. Delivery systems and/or instructions for use can also be included.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties. A number of embodiments have been described. Nevertheless, it will be understood that various modifications may be made. Accordingly, other embodiments are within the scope of the following claims.

### Examples

During normal immune responses, Tregs suppress T cell effector functions by generating immunosuppressive adenosine, cAMP, or anti-inflammatory cytokines (IL-10, TGF-β, IL-35), and by consuming IL-2. Furthermore, Tregs can cause effector T cell death via granzyme and perforin, and suppress activation of T cells by downregulating costimulatory molecules on antigen presenting cells (APCs) via CTLA-4. In addition to this indirect regulation, Tregs directly impair CD8+ T cell effector functions by compromising the release of lytic granules upon recognition of antigens on target cells. Although active regulation by Tregs plays a critical role in modulating host immunity, FoxP3+Tregs may negatively affect overall survival in the majority of solid tumors. In particular, decreased ratios of CD8+ T cells to Foxp3+ Treg cells among tumor-infiltrating lymphocytes directly correlate with poor prognosis in ovarian, breast, and gastric cancers. Despite its key regulation of anti-tumor immune responses, the molecular mechanisms underlying Treg-mediated immune suppression in the tumor microenvironment were unclear.

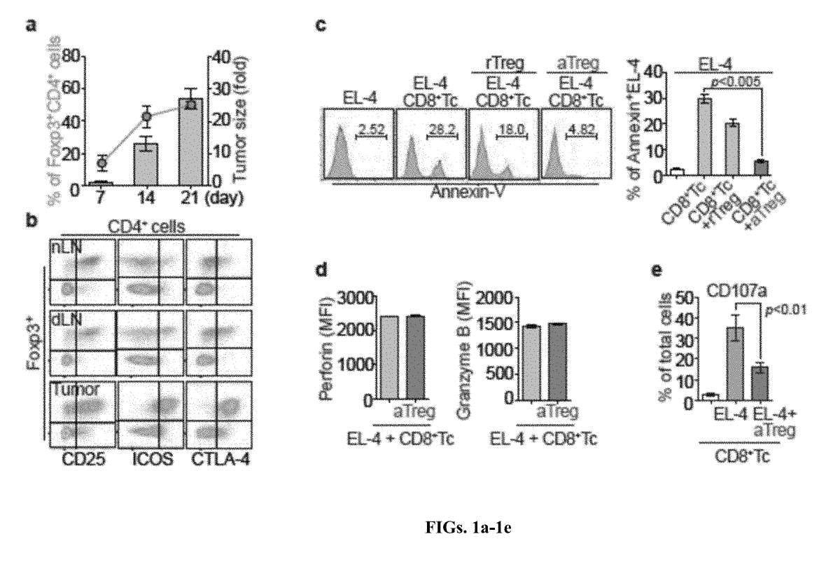

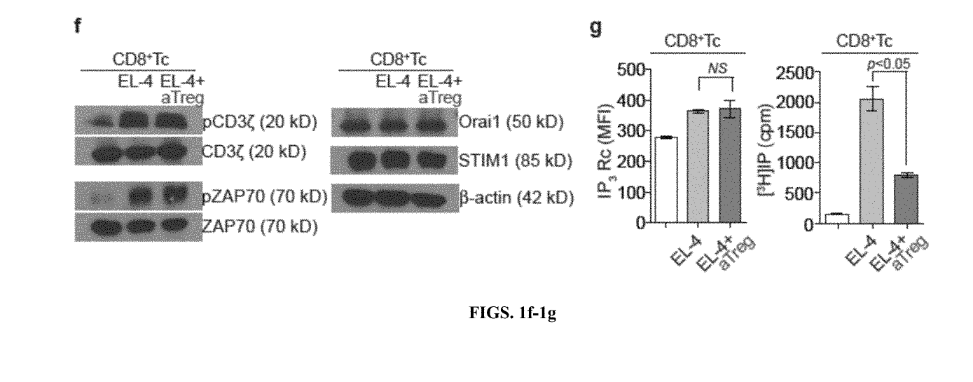

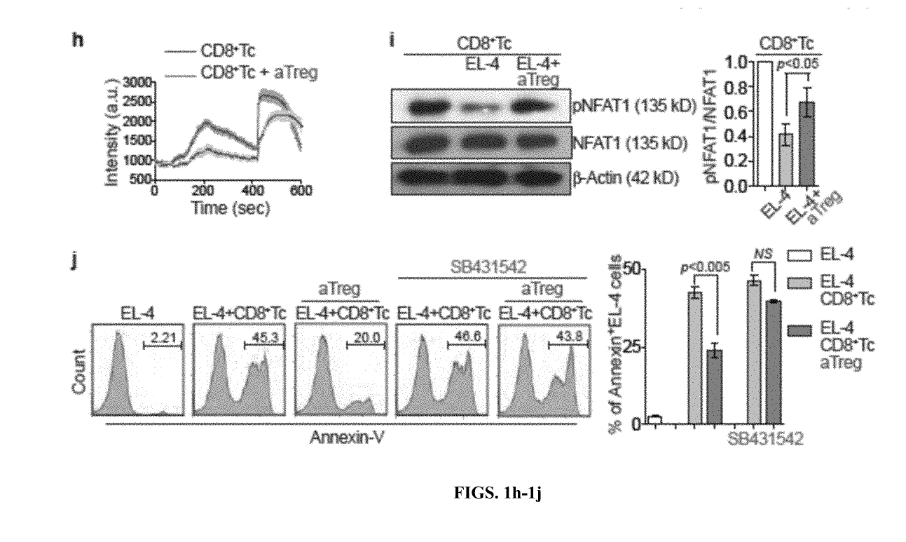

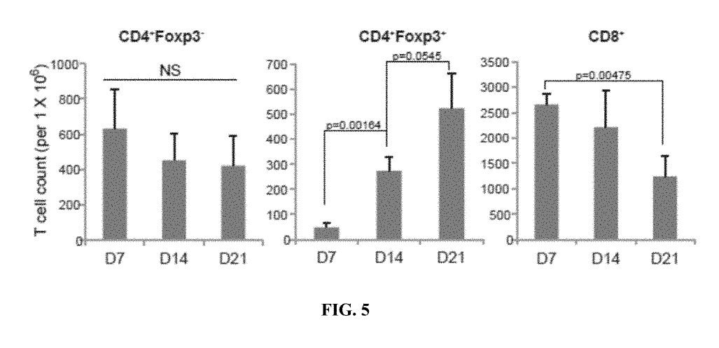

To determine the mechanisms of Treg-mediated immune suppression at the tumor site, the kinetics of Treg responses were measured in a mouse melanoma model. C57BL/6 mice injected intradermally in the ear with B16F10 tumor cells developed solid tumors with steady increases in both absolute numbers and ratios of Foxp3⁺CD4⁺ Treg cell population (FIG. 1(a)), FIG. 5). Flow cytometry analysis revealed that the majority of Tregs in the tumor showed activated and terminal effector Treg phenotypes (CD25^{high}ICOS^{high}CTLA-4^{high}) (FIG. 1(b)). The increased effector Treg cell counts in the tumor implied that these cells might play a key role in the loss of concomitant tumor immunity. To further study Treg-mediated suppression of CD8+ cytotoxic T cell (CD8+Tc) functions, OVA-loaded murine lymphoma EL-4 cells were co-cultured with CD8+Tc prepared from OT-I T cell receptor (TCR) transgenic mice, in the presence of resting naive (rTreg) or activated effector (aTreg) Tregs. CD8+Tc alone displayed significant cytotoxicity (Annexin-V⁺ or Propidium iodide⁺) against peptide-pulsed EL-4 (FIG. 1(c)). Preincubation of CD8+Tc with aTreg for 16 h completely abolished the tumoricidal functions of CD8+Tc, while incubation with rTreg had minimal effect on the levels of cytotoxicity (FIG. 1(c)). Importantly, expression of key effector molecules, perforin and granzyme B, was not changed by co-incubation of CD8+Tc with aTreg (FIG. 1(d)). Instead, the impaired cytotoxicity was mainly associated with a decrease in granule exocytosis as measured by surface expression of CD107a (FIG. 1(e)).

First, whether the observed suppression of granule exocytosis and cytotoxic functions of CD8+Tc could be attributed to the Treg-mediated inhibition of the TCR itself or TCR-proximal signals was investigated. Rapid tyrosine phosphorylation of CD3ζ in OT-I CD8+Tc upon incubation with OVA-loaded EL-4 cells was not suppressed by coincubation with aTreg (FIG. 1(f)). In addition, similar levels of ZAP-70 phosphorylation in CD8+Tc, both in the absence and presence of aTreg (FIG. 1(f), were detected. Store-operated Ca²⁺ entry is required for lymphocyte cytotoxicity. Orai1 and stromal interaction molecule 1 (STIM1) were identified as the molecular constituents of the calcium release-activated calcium (CRAC) channel in T cells. Therefore, whether Tregs suppress CD8+Tc lytic granule exocytosis by directly downregulating Orai1 and/or STIM1 expression was assessed. Again, co-incubation of CD8+Tc with aTreg did not affect Orai1 and STIM1 expression levels (FIG. 1(f)). These results show that Tregs have a minimal impact on TCR activation and CRAC expression.

TCR activation induces hydrolysis of phosphatidylinositol-(4,5)-bisphosphate into inositol-(1,4,5)-trisphosphate (IP3) by PLCγ, which induces the release of Ca²⁺ from ER stores by activating IP3-receptor. However, Tregs did not significantly change IP3-receptor expression in CD8+Tc (FIG. 1(g)). Surprisingly, Tregs caused a significant decrease in TCR-induced IP production in CD8+Tc, which led to a dramatic reduction of both TCR (first peak)- and ionomycin (second peak)-induced Ca²⁺ influx in CD8+Tc (FIG. 1h; note that increased intracellular Ca²⁺ in T cells by ionomycin also involves the generation of IP3) and NFAT1 dephosphorylation (an effector molecule downstream of Ca²⁺ signals in T cells) (FIG. 1i). Earlier studies reported that Treg cells directly suppress tumor-specific CD8+ T cell cytotoxicity through TGFβ signals. Importantly, it was shown that TGFβ suppresses Ca²⁺ influx in activated T cells through the inhibition of PLCγ-mediated interleukin-2 tyrosine kinase (ITK) activation. Similarly, aTreg-mediated suppression of CD8+Tc anti-tumor cytotoxicity was completely abolished by the TGFβ superfamily type I activin receptor-like kinase receptor inhibitor SB431542 (FIG. 1(j)), suggesting that the Treg-mediated suppression of tumor killing through intracellular Ca²⁺ signals is TGFβ-dependent.

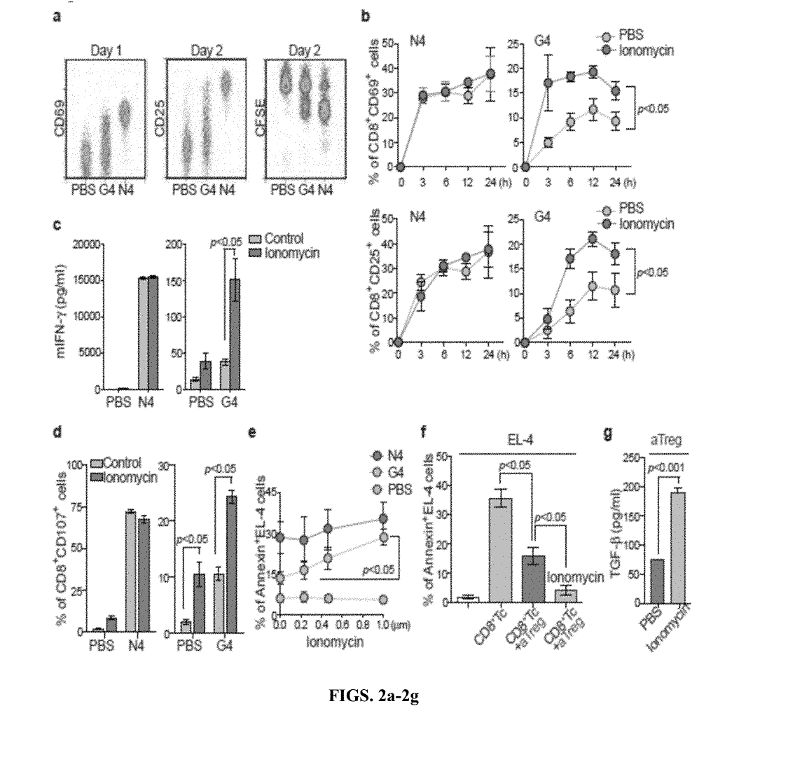

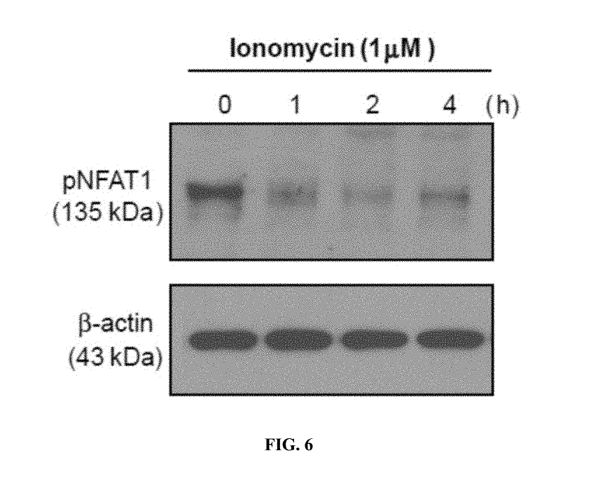

The finding that Tregs directly inhibit the TCR-dependent tumoricidal functions of CD8+Tc by suppressing IP3 production and Ca²⁺ influx suggests that strong intracellular Ca²⁺ signals in CD8+Tc can boost CTL functions at tumor sites. To study the effects of increased intracellular Ca²⁺ on T cell effector functions, the well characterized OT-I TCR transgenic mouse and altered peptide ligand (APL) system 6 (OVA257-264; N4: SIINFEKL (SEQ ID NO: 19) & G4: SIIGFEKL(SEQ ID NO; 20)) was used. G4 peptide is an OVA variant peptide with a single amino acid change at the highly exposed TCR contact sites on the pMHC complex and thus shows weaker affinities to TCR without altering the peptide affinity for MHCI (FIG. 2(a)). Ionomycin treatment of OT-I CD8+Tc significantly increased CD8 T cell activation, cytokine production, and degranulation in response to the weak-affinity antigen G4 (FIGS. 2(b), 2(c), & 2(d), FIG. 6). Consistently, ionomycin treatment improved the killing of G4-loaded EL-4 target cells to a level similar to that achieved against a high-affinity antigen (N4)-loaded EL-4 cell (FIG. 2(e)).

A significant limitation of cancer immunotherapy is that natural tumor antigens in general elicit relatively less robust T cell responses, in part because CTLs show low reactivity against tumor antigens while high-affinity T cells are rendered tolerant to these antigens. In addition, local immunosuppressive cells, such as Tregs, can further impair the effector functions of CTLs by inhibiting lytic granule release after recognition of the target cell. Therefore, the data provided herein led to the hypothesis that boosting intracellular Ca²⁺ signals in CTLs augments the lytic granule-dependent killing of target cells that express weak tumor antigens, even under the strong suppression by Tregs. To test this hypothesis, G4 peptide-loaded EL-4 cells were co-cultured with OT-I CD8+Tc in the presence of activated effector Tregs (aTregs). Preincubation of CD8+Tc with aTreg significantly reduced the tumoricidal effects (FIG. 2(f)). However, any improved cytotoxic activity of CD8+Tc after stimulation with ionomycin was not detected. Surprisingly, addition of ionomycin in the assay further suppressed CD8+Ta-mediated target cell killing (FIG. 2(f)). This suppression in the CD8+Tc response by ionomycin treatment was likely due to the simultaneous activation of Treg functions, as measured by the increased total TGF-β release from Tregs in the presence of ionomycin (FIG. 2(g)). These data strongly suggest that delivery of non-specific Ca²⁺ agonists to the tumor site will not provide the expected level of antitumor cytotoxicity by CD8+Tc. Instead, it may cause aberrant activation of local immune suppressive cells and thus a stronger suppression of CD8+Tc functions. Therefore, a more targeted and deliberate approach to selectively boost Ca²⁺ signals only in CD8+Tc at the tumor site was necessary.

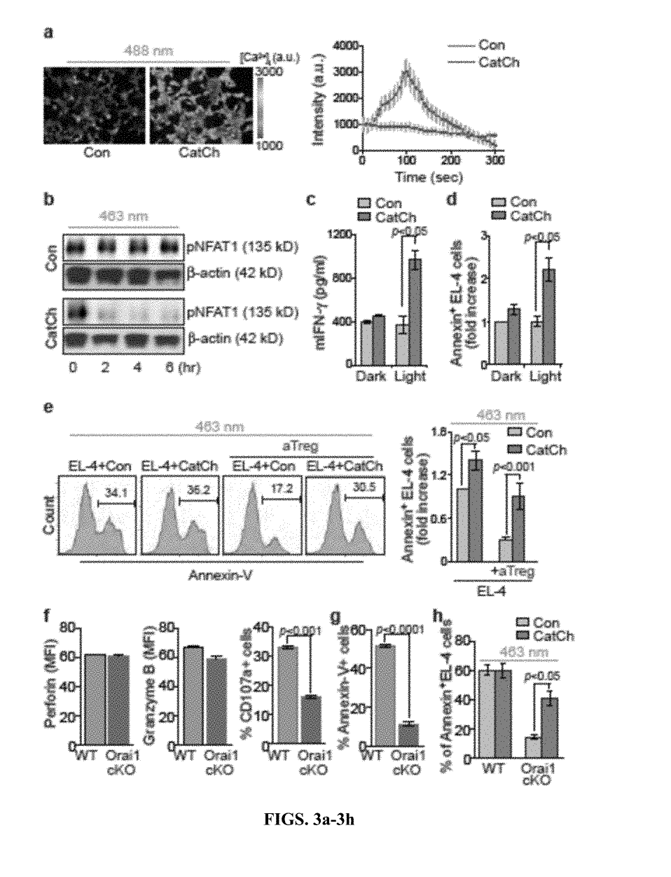

In hippocampal neurons, expression of CatCh, a new variant of channelrhodopsin, showed an accelerated membrane Ca²⁺ permeability, with 70-fold greater light sensitivity compared to that of wild-type channelrhodopsin 2, resulting in superior optogenetic control of intracellular Ca²⁺ influx. In this study, CatCh was used to selectively deliver Ca²⁺ activation signals only to the adoptively transferred CTLs in vivo, without interfering with endogenous Ca²⁺ signals in other cell types in the tumor microenvironment. To test the specific Ca²⁺ signals controlled by CatCh, [Ca²⁺ ]I was imaged in HEK293 cells transfected with CatCh. Fluorescence imaging of [Ca²⁺]i demonstrated that stimulation with green light (488±10 nm, 4.00 mW) was sufficient to drive prominent [Ca²⁺ ]i signals in CatCh-expressing cells but not in WT control cells, indicating the functional expression of CatCh (FIG. 3(a)). Furthermore, light stimulation of CatCh-expressing OT-I CD8+Tc was sufficient to drive prominent intracellular dephosphorylation of NFAT1 and cytokine production (IFNγ) in CatCh-expressing cells, but not in WT control cells or under dark conditions, indicating the feasibility of the remote activation of T cell Ca²⁺ signaling by light stimulation (FIGS. 3(b) & 3(c)). The ability of CatCh to control the cytotoxic functions of CD8+Tc was further confirmed by light stimulation of CatCh-expressing OT-I CD8+Tc during co-incubation 8 with G4-loaded EL-4 target cells. Optical stimulation of CatCh-expressing OT-I CD8+Tc yielded a significant increase in target cell killing comparing to dark condition (FIG. 3(d)). An important advantage of CatCh is its ability to deliver highly selective Ca²⁺ stimulation in CTLs and thus boost their effector functions without activating other immunosuppressive cells, such as Tregs, at the tumor site. This ability was demonstrated by light stimulation of CatCh-expressing OT-I CD8+Tc during co-incubation with G4-loaded EL-4 target cells in the presence of aTregs. Light activation of CatCh-expressing OT-I CD8+Tc significantly increased killing of target cells, allowing them to successfully overcome the Treg-mediated suppression (FIG. 3e).

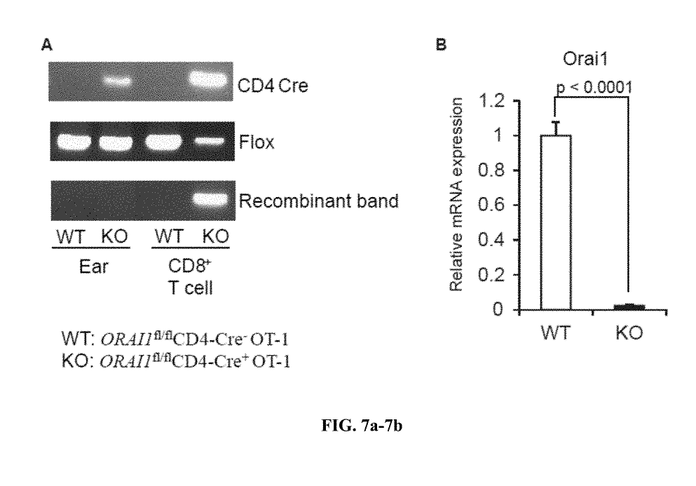

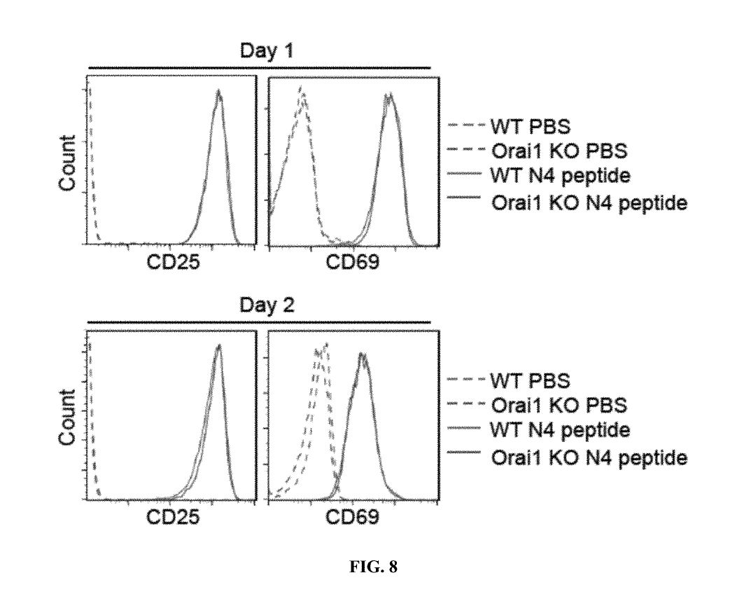

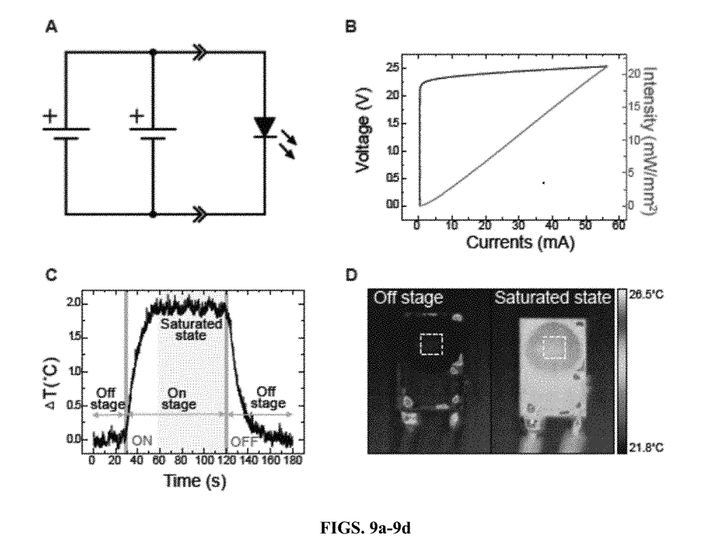

TCR activation induces IP3-mediated release of Ca²⁺ from ER stores. After depletion of these Ca²⁺ stores, the Ca^{t}′ sensor STIM1 activates a highly selective Orai1 Ca²⁺ channel. This channel is located at the plasma membrane and is responsible for store-operated Ca²⁺ entry from outside of the T cell. An increase in intracellular Ca²⁺ in T cells by ionomycin also involves the generation of IP3, depletion of the intracellular Ca²⁺ stores and activation of the Orai1 channel. In contrast, CatCh is a cell-membrane calcium channel that can bypass the IP3 generation and the depletion of Ca²⁺ stores and induce Ca²⁺ influx directly through the membrane. Therefore, the different modes of Ca²⁺ signaling may induce different downstream signaling responses in T cells. To test whether induction of Ca²⁺ influx by light stimulation of CatCh can recapitulate the physiological functions of Orai1 channels during CTL killing, T cell specific Orai1 conditional knockout (KO) mice were generated by crossing Orai1fl/fl mice [30-33] to Cd4− Cre mice. Deletion of Orai1 gene expression in CD8+ T cells was validated by PCR (FIG. 7). CD8+ T cell differentiation, activation, and expression of effector 9 molecules (perforin and granzyme B) in Orai1 KO CD8+Tc were comparable to those in WT CD8+ T cells (FIG. 3(f) & FIG. 8). However, lytic granule exocytosis and killing of target EL-4 cells were severely altered in Orai1 KO CD8+Tc (FIGS. 3(f) and 3(g)). Importantly, light stimulation of CatCh-expressing Orai1 KO CD8+Tc successfully restored the cytotoxic function of CTLs (FIG. 7). These results, in combination with the earlier Treg data, support the conclusion that CatCh can be functionally expressed in CD8+ T cells to allow photoactivatable control of Ca²⁺ signals and boost T cell mediated tumor killing using remote light stimulation within the immunosuppressive tumor microenvironment.

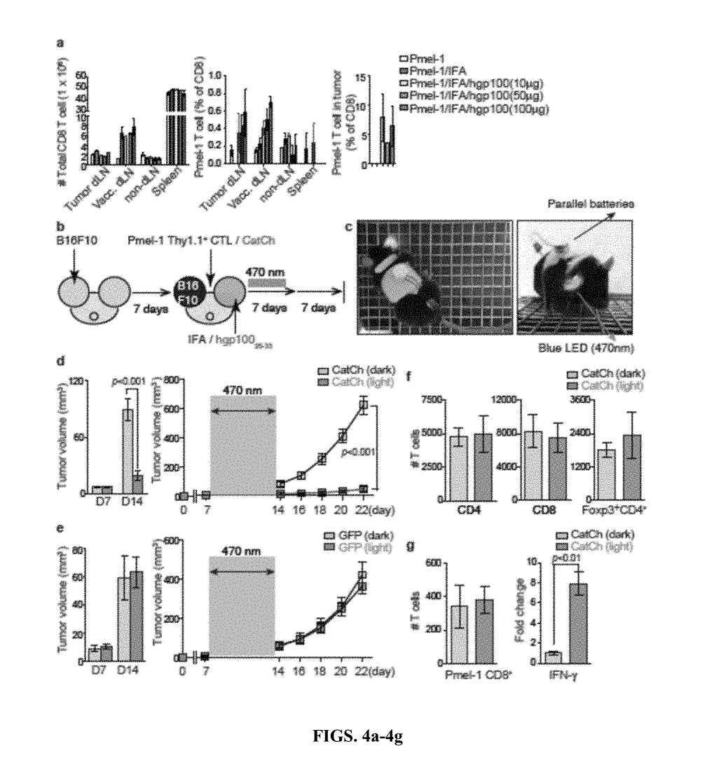

To demonstrate the clinical implications of CatCh-mediated immune boosts, the ability of CatCh to enhance the cytotoxicity of adoptively transferred tumor-specific CD8+Tc and to improve antigen-specific tumor regression was examined. In this study, a Pmel-1 TCR transgenic mouse model, one of the well characterized mouse tumor models for the low immunogenicity, which expresses the Vα1Vβ13 TCR that recognizes an H-2Db-restricted epitope corresponding to amino acids 25-33 of mouse gp100 (mgp100) on the B16 melanoma cells was used. B16 melanoma cells grow at a normal rate in Pmel-1 mice despite the presence of overwhelming numbers of mgp100-specific CD8+ T cells (FIG. 4(a)). Furthermore, antigen-specific vaccination with a mgp100 altered peptide ligand (e.g., human gp100; hgp100) was not sufficient to improve the antitumor effects of adoptively transferred Pmel-1 T cells against B16 tumors due to an increase in the local CD4+FoxP3+ Treg cell population.

To determine whether the activation of adoptively transferred CD8+Tc were enhanced in order to treat established solid tumors, it was demonstrated that light stimulation of CatCh could deliver highly selective Ca²⁺ stimulation in Pmel-1 T cells and thus boost their effector functions under the Treg-mediated suppression. Light activation of CatCh-expressing Pmel-1 CD8⁺ Tc significantly increased killing of hgp100₂₅₋₃₃ peptide (KVPRNQDWL) (SEQ ID NO: 18)-loaded B16 target cells, allowing them to successfully overcome the Treg-mediated suppression (FIG. 11(a)). For in vivo experiments, Pmel-1 CD8+Tc expressing CatCh were adoptively transferred into C57BL/6 mice bearing subcutaneous B16 tumors established for 7 days, followed by vaccination with hgp100₂₅₋₃₃ peptide (KVPRNQDWL)(SEQ ID NO: 18) (FIG. 4(b) and FIG. 11(b)). Subsequently, the visible and palpable tumor area was illuminated for 7 days, and tumor growth was measured for an additional 7 days without illumination. For long-term in vivo light exposure in freely moving animals, a battery-powered wireless blue light emitting diode (LED; 470 nm) was glued to the mouse ear skin (FIG. 4(c) and FIG. 9). The peak light output during light stimulation was determined empirically at 0.1-5 mW/mm2 (470 nm, 3.67 mW/mm² on average) at the surface of LED. Dark mice were treated equally for a total of 21 days without light stimulation. Localized light stimulation dramatically decreased tumor growth (FIG. 4(d)). Light stimulation of the mice that received GFP-transfected Pmel-1 CD8+Tc did not alter tumor growth, indicating that the effect of 470-nm LED light alone was not detrimental to the tumor cells (FIG. 4(e)). Flow cytometry analyses of B16 tumors confirmed that local light stimulation did not significantly change total T cell numbers nor intratumoral Pmel-1 CD8+Tc infiltration, comparing to the dark mice (FIGS. 4(f) & 4(g)). However, local light activation substantially increased the level of IFN-γ expression (FIG. 4(g)), showing that the enhanced Ca²⁺ signals in CatChexpressing CTLs by light stimulation can improve the cytotoxic functions of CD8+Tc responses by promoting local effector functions and antitumor activity.

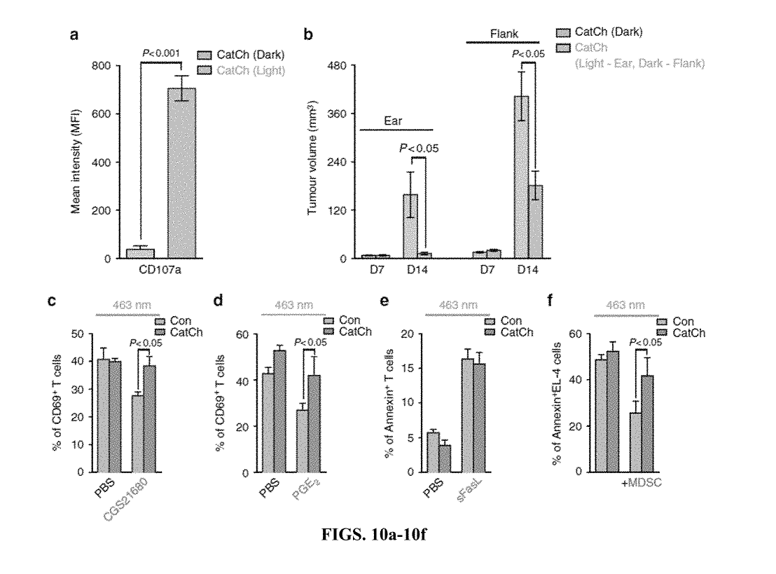

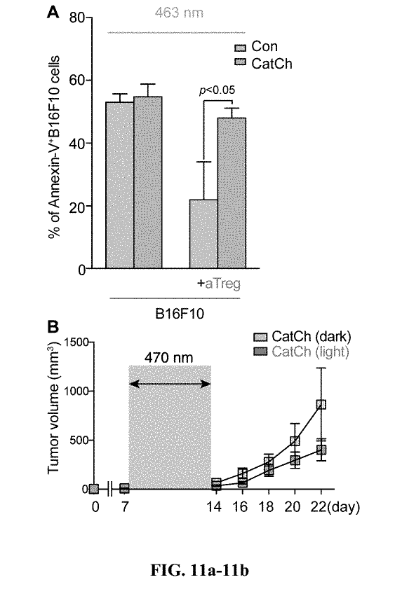

To further test whether localized light activation of CatCh-expressing CD8⁺ Tc induces systemic effects and thus controls non-illuminated tumor growth at a distal secondary site, Pmel-1 CD8⁺ Tc expressing CatCh were adoptively transferred into C57BL/6 mice bearing two subcutaneous B16 tumors at the ear and flank, followed by vaccination with hgp100peptide. Subsequently, the visible and palpable tumor area at the ear was illuminated for 7 days, and tumor growth was measured at the flank. Localized light activation dramatically increased the cell surface expression of CD107a on Pmel-1 CD8⁺ Tc at the illumination site (FIG. 10a), suggesting that the enhanced Ca²⁺ signals in CatCh-expressing CTLs by light stimulation can not only improve the cytokine production (FIG. 4g), but also promote cytotoxic functions of CD8⁺ Tc responses by inducing granule exocytosis. The enhanced local CD8⁺ Tc effector functions by light stimulation at the ear significantly decreased tumor growth both at the illuminated ear and non-illuminated flank (FIG. 10b). These results strongly suggest that light stimulation of local CD8⁺ Tc function could trigger systemic effects and induce anti-tumor responses outside the illumination field. Clinically, this is relevant for treatment of solid tumors established in their metastases.

Growing evidence suggests that Tregs do not use only one universal mechanism of immune suppression at the tumor microenvironment, but rather execute suppressive functions through several different modes. Therefore it is possible that the improved antitumor activity of CatCh-expressing CD8⁺ Tc by light stimulation in vivo can mediate a combination of multiple processes other than direct induction of lytic granule exocytosis from CTLs seen in in vitro assays. Indeed, several mechanisms have been proposed for the Treg-mediated direct suppression of CD8⁺ T cell anti-tumor effector functions, which include Fas/FasL-dependent T cell apoptosis and suppression of effector T cells by releasing adenosine (Ado) and PGE₂. This possibility was addressed by light stimulation of CatCh-expressing OT-I CD8⁺ Tc in the presence of soluble Fas ligand (sFasL), CGS21680 (A_{2A} receptor agonist), or PGE₂. Light activation allowed CatCh-expressing OT-I CD8⁺ Tc to successfully overcome A_{2A} receptor- and PGE₂-mediated suppression of T cell activation in the presence of Ag-loaded APC, but failed to reverse sFasL-mediated T cell apoptosis (FIG. 10c-e).

In addition to Treg, tumor-resident myeloid-derived suppressor cells (MDSCs) can further counteract proper CTL effector functions at the tumor microenvironment. To test whether optical stimulation of Ca²⁺ signals can reverse MDSC-induced CTL killing inefficiency, CatCh-expressing OT-I CD8⁺ Tc were stimulated with light during co-incubation with N4-loaded EL-4 target cells in the presence of MDSCs. Light activation of CatCh-expressing OT-I CD8⁺ Tc significantly increased killing of target cells, allowing them to partially overcome the MDSC-mediated suppression (FIG. 10f). Therefore, this data shows that light stimulation of CatCh in vivo not only improves the lytic granule exocytosis (FIG. 10a), but also boosts suppressed CTL responses by multiple inhibitory factors derived from both Treg and MDSC.

In conclusion, the data provided herein show that Treg-mediated suppression of CTL killing is not induced by changes in TCR-proximal signals, but is mainly mediated by TGFβ-induced inhibition of IP3 production, which directly decreases intracellular Ca²⁺ responses and T cell degranulation. Highly selective optical control of Ca²⁺ signals in CTLs at the tumor site was sufficient to overcome Treg-induced immunosuppression and dramatically improve the efficacy of adoptive T cell transfer immunotherapy in vivo. Further, the use of light to control immune reactions avoids the need for direct physical contact with the tissue and therefore any interference with normal functions. Importantly, light offers outstanding spatial resolution, allowing access to specific cellular subtypes and even the smallest subcellular domains. Therefore, the optogenetic approaches described in this study allow the remote control of CTL effector functions at tumor sites with outstanding specificity and temporospatial resolution.

### Methods

### Mice

C57BL/6J, TCR-transgenic OT-I mice and CD4-cre mice were purchased from the Jackson Laboratory. The Orai1 floxed (C57BL/6 background) mice were a generous gift from Drs. Yousang Gwack and Sonal Srikanth. Orai1fl/fl mice were bred to CD4-Cre mice to yield T cell-specific Orai1-deficient mice. Orai1fl/flCD4-Cre mice were bred to TCRtransgenic OT-I mice to yield Orai1fl/flCD4-Cre OT-1 mice. The mice were housed under pathogen-free conditions. All mouse experiments were approved by the University Committee on Animal Resources at the University of Rochester.

### Cell Lines Culture

EL-4 mouse lymphoma cell line and B16F10 mouse melanoma cell line were cultured in DMEM supplemented with 10% FCS and penicillin-streptomycin. 293T cells and Phoenix cells were cultured in DMEM supplemented with 10% FBS, 100 U/ml penicillin, 100 μg/ml streptomycin, 2 mM L-glutamine, 20 mM HEPES buffer, 1% MEM Non-Essential Amino Acid and 50 μM β-mercaptoethanol.

### B16F10 Tumor Model

2×10⁵B16F10 cells in 10 μl PBS were intradermally injected into one ear pinna of a recipient C57BL/6 mouse. Tumor growth was monitored every week from day 7 after tumor injection. Tumor volume was calculated as width×length×0.52. The mice were sacrificed on days 7, 14 and 21 post tumor injection. To analyze tumor samples, mouse ear tumors were cut into small pieces and digested with collagenase/dispase (Roche (Basel, Switzerland), 10269638001) at 37° C. for 30 min.

### Antibodies

The following antibodies used for flow cytometry were purchased from eBioscience (Waltham, Mass.), BD Biosciences (San Jose, Calif.) or BioLegend (San Diego, Calif.): anti-CD4 (V450, RM4-5), anti-CD8 (PerCP, 53-6.7), anti-Thy1.1 (PE, HIS51), anti-Foxp3 (APC, FJK-16S), anti-CD25 (PE, PC61.5), anti-ICOS (PE/CyS, 7E17G9), anti-CTLA-4 (PE/Cy7, UC10-4B9), Annexin-V (APC), anti-Perforin (APC, eBioOMAK-D), anti-Granzyme B (PE/Cy7, NGZB), anti-CD107a (Alexa-Fluor 647, 1D4B) and anti-CD69 (PE/Cy7, HI.2F3). The following antibodies were used for Western blot analysis: anti-phospho-CD3-(Sigma (St. Louis, Mo.), SAB4200334), anti-CD3-(Invitrogen (Carlsbad, Calif.), MA1-10188), anti-phospho-ZAP-70 (Cell Signaling (Danvers, Mass.), #2717), anti-ZAP-70 (Cell signaling, #2705), anti-Orai1 (ProSci (Poway, Calif.), PM-5207), anti-Stiml (Cell Signaling, #5668), anti-β-Actin (Sigma, A2228), anti-phospho-NFAT1 (Santa Cruz (Dallas, Tex.), SC-32994) and anti-NFAT1 (Cell Signaling, #5862). Anti-CD3 (BD Biosciences, 553057) and anti-CD28 (BioLegend, #102102) were used for calcium imaging and the antibodies were cross-linked with goat anti-hamster IgG (MP Biomedicals (Santa Ana, Calif.), #855397).

### T Cell Purification and Activation

OT-1 CD8+ T cells were purified from single-cell suspensions of lymph nodes of OT-1 mice. Single-cell suspensions were prepared by mechanical disruption using a cell strainer. OT-1 CD8+ T cells were then enriched by magnetic-bead depletion with mouse MHC Class II antibody (M5/114) and anti-CD4 antibody (GK1.5), followed by Low14 Tox-M Rabbit Complement (Cedarlane (Burlington, N.C.) invitro, CL3051) and sheep anti-rat IgG magnetic beads (Invitrogen, 11035). For effector T cell differentiation, cells were stimulated with OVA peptide (4 μg/ml) and irradiated splenocytes for 5 days in IL-2 (50 U/ml) containing media. Regulatory T cells were purified from single-cell suspensions of lymph nodes and spleens of 8-12 week-old C57BL/6J mice and enriched with a CD4+CD25+ Regulatory T Cell Isolation Kit (Miltenyi Biotec (Bergisch Gladbach, Germany), 130-091-041) according to the manufacturer's protocol. To generate activated Tregs, purified Tregs were stimulated with plate-bound anti-CD3 (3 μg/ml) and anti-CD28 (3 μg/ml) for 48 h in IL-2 (20 U/ml) containing media.

### Cytotoxicity Assay

OT-1 CTLs were co-cultured without with or without Tregs in the presence of IL-2 (20 U/ml) for 16 h. To measure OT-1 CTL cytotoxicity, fluorescent dye (CF SE or PKH-26)-labeled target EL4 cells were pulsed with Ova peptide (N4 or G4) for 2 h. After washing EL-4 cells three times with PBS, EL-4 cells were added to OT-1 CTLs with or without Tregs. After 5 h, cytotoxicity was measured by flow cytometry after Annexin-V staining.

### Western Blot

CFSE-labeled OT-1 CTLs were isolated using fluorescence-activated cell sorting (FACS) from unlabeled EL-4 and aTreg in co-culture experiments. For protein extraction, cells were lysed in RIPA buffer (Thermo Scientific (Waltham, Mass.), #89900) and 1× Halt protease & phosphatase inhibitor cocktail (Thermo Fisher Scientific, #78440). Electrophoresis was performed on PAGEr Gold Precast Gels (Lonza (Basel, Switzerland), #58522), and proteins were transferred 15 to nitrocellulose membranes (Thermo scientific, #88018). After blocking with 5% nonfat dry milk, the blots were incubated overnight with the different primary antibodies used. All secondary antibodies were conjugated with horseradish peroxidase (HRP). SuperSignal West pico (Thermo scientific, 32106) and Supersignal West Femto (Pierce (Carlsbad, Calif.), #34095) were used to detect HRP on immunoblots with X-ray film (Pierce, #34090).

### Inositol Phosphate Assay

After co-culture with or without aTregs, OT-1 CTLs were labeled by adding 4 μCi of [³H] inositol for 24 h in inositol-free F-10 media. After labeling, LiCl was added directly to the labeling media at a final concentration of 10 mM. After 10 min, peptide pulsed EL-4 were added to the plate. The final volume of each well was 1 ml. The plates were placed back in to the incubator at 37° C. for 30 min. The cells were then washed were washed twice with cold PBS. Ice-cold 50 mM formic acid, 1 ml, was added to the cells, which were placed on ice for 30 min. After the incubation, the lysates were applied to Dowex AX1-X8 columns and allowed to flow all the way through the column. The columns were washed with 50 and 100 mM formic acid, followed by elution of the inositol phosphate (IP)-containing fraction with 3 ml of 1.2 M ammonium formate/0.1 M formic acid. The eluted fraction was mixed with 10 ml of scintillation fluid and measured by liquid scintillation counting.

### Calcium Imaging

HEK293T cells were transfected with the pcDNA3.1-CatCh-mCherry plasmid using Lipofectamine 2000 (Invitrogen, 11668-030) on a Delta T culture dish (Bioptechs (Butler, Pa.), 16 04200417C). PKH-26-labeled OT-1 CTLs were co-cultured with or without pre-activated Tregs before TCR stimulation. Calcium imaging was performed as described previously.

### CatCh Expression in T Cells

The CatCh nucleic acid sequence was cloned into the pMIGR-GFP vector. CatCh and GFP control retroviruses were generated using the Phoenix-ecotropic packaging cell line. For retroviral transductions, Phoenix cells were transfected with the above plasmids to produce retroviruses using the calcium phosphate transfection method. Virus-containing supernatant was collected at 2 and 3 d after transfection. OT-1 CD8+ T cells or Pmel-1 CD8+ T cells were transduced on day 1 after activation in the presence of 8 mg/ml polybrene. The cells were sorted based on GFP expression.

### Light Stimulation

Approximately 2×10⁵ B16F10 cells in 10 μl PBS were intradermally injected into one ear pinna of a recipient C57BL/6 mouse. Tumor size was measured at day 7, and mice with a similar size tumor were selected for experiments. Mice were vaccinated by intradermal injection of mouse ear skin with 10 μl PBS/IFA (Incomplete Freund's adjuvant) emulsion containing 10 μg of hgp100₂₅₋₃₃ peptide (KVPRNQDWL)(SEQ ID NO: 18). Then, 2×10⁶ CatCh-expressing Pmel-1 CTLs or GFP-expressing Pmel-1 CTLs were injected either via tail vein or retro-orbital injection. Blue LED emitters (Future electronics (Quebec, CA), LXML-PB01-0030) were attached to the tumor site with glue. Lithium batteries were connected to blue 17 LED emitters and replaced daily. The design of the Battery-powered wireless LED is described in FIG. 9. For in vitro light stimulation, an optical fiber was installed in CO2 incubator. The fiber was coupled to an LED system (Doric Lens, LEDRV 2CH 1000) through a blue LED module (Doric Lens (Quebec, CA), LEDC1-B_FC). The peak light output during 463-nm light stimulation was estimated to be 17 mW/cm2 at the tip of the optic fiber. Blue light pulses (1 Hz, 500 ms on, 500 ms off) were delivered by Optogenetics TTL Pulse generator (Doric Lens, OTPG 4). The cells were cultured in glass bottom 96-well plate (MatTek (Ashland, Mass.), P96G-1.5-5-F) for light illumination.

### RT-PCR

Total RNA was extracted from collagenase/dispase-treated tumor samples using RNA isolation kit (Qiagen (Hilden, Germany) #74104). cDNA was synthesized from total RNA using Superscript III First-Strand cDNA synthesis kit (Invitrogen, #18080051). Real-time PCR was performed using an iCycler IQ5 system and SsoFast EvaGreen Supermix (Bio-Rad (Hercules, Calif.), #1725201) using the following primer pairs: GAPDH (forward: 5′-CATGGCCTTCCGTGTTCCTA-3′(SEQ ID NO: 2) and reverse: 5′-CCTGCTTCACCACCTTCTTGAT-3) (SEQ ID NO: 3), perforin (forward: 5′-GAGAAGACCTATCAGGACCA-3′(SEQ ID NO: 4) and reverse: 5′-AGCCTGTGGTAAGCATG-3′) (SEQ ID NO: 5), granzyme B (forward: 5′-CCTCCTGCTACTGCTGAC-3′ (SEQ ID NO: 6) and reverse: 5′-GTCAGCACAAAGTCCTCTC-3′) (SEQ ID NO: 7), IL-2 (forward: 5′-CCTGAGCAGGATGGAGAATTACA-3′(SEQ ID NO: 8) and reverse: 5′-TCCAGAACATGCCGCAGAG-3′) (SEQ ID NO: 9), IFN-g (forward: 5′-GGATGCATTCATGAGTATTGC-3′ (SEQ ID NO: 10) and reverse: 5′-CCTTTTCCGCTTCCTGAGG-3′) (SEQ ID NO: 11) and TNF-α (forward: 5′-GACGTGGAACTGGCAGAAGAG-3′(SEQ ID NO: 12) and reverse: 5′-TTGGTGGTTTGTGAGTGTGAG-3′) (SEQ ID NO: 13). β-actin (forward: 5′-GGCTGTATTCCCCTCCATCG-3′ (SEQ ID NO: 14) and reverse: 5′-CCAGTTGGTAACAATGCCATGT-3′) (SEQ ID NO: 15) and Orai1 (forward: 5′-GATCGGCCAGAGTTACTCCG-3′ (SEQ ID NO: 16) and reverse: 5′-TGGGTAGTCATGGTCTGTGTC-3′) (SEQ ID NO: 17).

### Statistical Analysis

All statistical tests were done with GraphPad Prism and Jmp Software (SAS). Statistical analysis was performed using One-Way ANOVA with a Bonferonni post-test, unpaired ttest, and Mann-Whitney when appropriate. Differences were considered significant when p values were <0.05.

### CatCh Polypeptide Sequence

(SEQ ID NO: 1)
MDYGGALSAVGRELLFVTNPVVVNGSVLVPEDQCYCAGWIESRGTNG
AQTASNVLQWLAAGFSILLLMFYAYQTWKSTCGWEEIYVCAIEMVKV
ILEFFFEFKNPSMLYLATGHRVQWLRYAEWLLTCPVICIHLSNLTGL
SNDYSRRTMGLLVSDIGTIVWGATSAMATGYVKVIFFCLGLCYGANT
FFHAAKAYIEGYHTVPKGRCRQVVTGMAWLFFVSWGMFPILFILGPE
GFGVLSVYGSTVGHTIIDLMSKNCWGLLGHYLRVLIHEHILIEGDIR
KTTKLNIGGTEIEVETLVEDEAEAGAV

What is claimed is:

1. A modified T cell comprising an exogenous nucleic acid encoding a calcium translocating channelrhodopsin (CatCh) polypeptide.

2. The modified T cell of claim 1, wherein the modified T cell is an activated T cell.

3. The modified T cell of claim 1, wherein the modified T cell is a CD8+ T cell.

4. The modified T cell of claim 1, wherein the T cell is further modified to express a chimeric antigen receptor (CAR).

5. The modified T cell of claim 1, wherein the nucleic acid is in a vector.

6. The modified T cell of claim 1, wherein the nucleic acid encoding the CatCh polypeptide is under the control of an inducible promoter.

7. The modified T cell of claim 1, wherein the calcium translocating CatCh polypeptide comprises SEQ ID NO: 1.

8. A non-human animal comprising the modified T cell of claim 1.

9. A method of treating cancer in a subject comprising administering to the subject one or more modified T cells of claim 1 and exposing the one or more modified cells in the subject to a visible light source.

10. The method of claim 9, wherein the cancer is selected from the group consisting of skin cancer, colon cancer, breast cancer, prostate cancer, esophageal cancer, rectal cancer, throat cancer, lung cancer and stomach cancer.

11. The method of claim 9, wherein the one or more modified T cells are derived from one or more T cells removed from the subject and transfected ex vivo with the exogenous nucleic acid encoding a CatCh polypeptide.

12. The method of claim 9, wherein the one or more modified T cells are directly administered to a tumor in the subject.

13. The method of claim 9, wherein the one or more modified T cells are administered to the subject at a surgical site.

14. The method of claim 9, wherein exposing the one or more modified T cells in the subject to a visible light source increases the immune response of the subject.

15. The method of claim 9, wherein exposing the one or more modified T cells in the subject to a visible light source promotes T cell-mediated tumor killing.

16. The method of claim 9, wherein exposing the one or more modified T cells in the subject to a visible light source selectively stimulates Ca²⁺ production in the one or more modified T cells and does not activate immunosuppressive T cells in the subject.

17. The method of claim 9, wherein the visible light source is a laser or a light emitting diode.

18. The method of claim 17, wherein the visible light source emits light at a wavelength of about 450 to 515 nm.

19. The method of claim 9, wherein the subject is a human subject.


SEQUENCE LISTING (20 sequences)

SEQ ID NO: 1 - protein, 309 aa, Artificial sequence
  Synthetic construct
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVICIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAV

SEQ ID NO: 2 - DNA, 20 bp, Artificial sequence
  Synthetic construct
catggccttccgtgttccta

SEQ ID NO: 3 - DNA, 22 bp, Artificial sequence
  Synthetic construct
cctgcttcaccaccttcttgat

SEQ ID NO: 4 - DNA, 20 bp, Artificial sequence
  Synthetic construct
gagaagacctatcaggacca

SEQ ID NO: 5 - DNA, 17 bp, Artificial sequence
  Synthetic construct
agcctgtggtaagcatg

SEQ ID NO: 6 - DNA, 18 bp, Artificial sequence
  Synthetic construct
cctcctgctactgctgac

SEQ ID NO: 7 - DNA, 19 bp, Artificial sequence
  Synthetic construct
gtcagcacaaagtcctctc

SEQ ID NO: 8 - DNA, 23 bp, Artificial sequence
  Synthetic construct
cctgagcaggatggagaattaca

SEQ ID NO: 9 - DNA, 19 bp, Artificial sequence
  Synthetic construct
tccagaacatgccgcagag

SEQ ID NO: 10 - DNA, 21 bp, Artificial sequence
  Synthetic construct
ggatgcattcatgagtattgc

SEQ ID NO: 11 - DNA, 19 bp, Artificial sequence
  Synthetic construct
ccttttccgcttcctgagg

SEQ ID NO: 12 - DNA, 21 bp, Artificial sequence
  Synthetic construct
gacgtggaactggcagaagag

SEQ ID NO: 13 - DNA, 21 bp, Artificial sequence
  Synthetic construct
ttggtggtttgtgagtgtgag

SEQ ID NO: 14 - DNA, 20 bp, Artificial sequence
  Synthetic construct
ggctgtattcccctccatcg

SEQ ID NO: 15 - DNA, 22 bp, Artificial sequence
  Synthetic construct
ccagttggtaacaatgccatgt

SEQ ID NO: 16 - DNA, 20 bp, Artificial sequence
  Synthetic construct
gatcggccagagttactccg

SEQ ID NO: 17 - DNA, 21 bp, Artificial sequence
  Synthetic construct
tgggtagtcatggtctgtgtc

SEQ ID NO: 18 - protein, 9 aa, Artificial sequence
  Synthetic construct
KVPRNQDWL

SEQ ID NO: 19 - protein, 8 aa, Artificial sequence
  Synthetic construct
SIINFEKL

SEQ ID NO: 20 - protein, 8 aa, Artificial sequence
  Synthetic construct
SIIGFEKL